(19) United States
(12) Patent Application Publication
(10) Pub. No.: US 2023/0380456 A1
(43) Pub. Date: Nov. 30, 2023

(54) SYSTEMS AND METHODS FOR UV TREATMENT OF A VISCOUS FLUID

(71) Applicant: PepsiCo, Inc., Purchase, NY (US)

(72) Inventors: Yuqian LOU, Warren, NJ (US); Thaddeus PESCE, White Plains, NY (US); Prashant POKHREL, Spring Valley, NY (US)

(21) Appl. No.: 17/804,044

(22) Filed: May 25, 2022

(51) Int. Cl. A23L 3/28; A23L 5/30; A23L 27/30

(52) U.S. Cl. CPC A23L 3/28; A23L 5/36; A23L 27/33

(57) ABSTRACT

Some embodiments are directed to systems and methods for treating a viscous fluid. The viscous liquid may be a liquid sugar. The viscous fluid may have a viscosity of at least 50 cP. The system may include one or more mixers configured to receive the viscous fluid and to generate turbulent flow of the viscous fluid; and one or more UV chambers configured to receive the viscous fluid from the mixer and to expose the viscous fluid to a dose of UV light, the dose being at least 250 mJ/cm². The system may deliver a total dose of UV light of about 500 mJ/cm² such that bacteria and other contaminants are reduced or eliminated.

### BACKGROUND

The present disclosure relates to systems and methods for treating a viscous fluid and/or a fluid with low ultraviolet transmittance (UVT) with ultraviolet (UV) light. More specifically, the present disclosure relates to systems and methods for reducing contaminants in a viscous fluid using UV light.

### BRIEF SUMMARY

Some embodiments are directed to a system for treating a viscous fluid. In some embodiments, the system includes a mixer configured to receive the viscous fluid and to generate turbulent flow of the viscous fluid. In some embodiments, the system includes a UV chamber configured to receive the viscous fluid from the mixer and to expose the viscous fluid to a dose of UV light. In some embodiments, the dose is about 250 mJ/cm².

In some embodiments, the viscous fluid has a viscosity of at least 50 cP. In some embodiments, the viscous fluid has a viscosity of 50 cP to 250 cP.

In some embodiments, the system further includes a second mixer configured to receive the viscous fluid from the UV chamber and to generate turbulent flow of the viscous fluid. In some embodiments, the system further includes a second UV chamber configured to receive the viscous fluid from the second mixer and to expose the viscous fluid to a second dose of UV light to produce a treated viscous fluid. In some embodiments, the second dose being at least 250 mJ/cm².

In some embodiments, the UV chamber is configured to expose the viscous fluid to UV light for about 1 second to about 5 seconds, and the second UV chamber is configured to expose the viscous fluid UV light for about 1 second to about 5 seconds.

In some embodiments, the mixer and the second mixer are each a static mixer.

In some embodiments, the system is configured to treat the viscous fluid to form a treated fluid having an acrylamide content of less than 2 μg/kg, a total furan content of less than μg/kg, a hydroxy methyl furan content of less than 5 ppm, and a 4-methylimidazole content of less than 0.0100 mg/kg, and a furfuryl alcohol content of less than 0.5 mg/kg.

In some embodiments, the viscous fluid is a liquid sugar, and the system further includes a melting tank. In some embodiments, the melting tank can form the liquid sugar from water and sugar.

In some embodiments, the viscous fluid is a liquid sugar having a sugar content from 60 Brix to 70 Brix. In some embodiments, the viscous fluid is a liquid sugar having a sugar content from 67 Brix to 68 Brix. In some embodiments, the viscous fluid has an ultraviolet transmittance of about 25% to about 50%.

In some embodiments, the system maintains the viscous fluid at a Reynolds number of at least 2200 through the UV chamber.

In some embodiments, the system further includes a melting tank configured to form the viscous fluid. In some embodiments, the viscous fluid is a liquid sugar formed from water and sugar, and the liquid sugar has a sugar content from 60 Brix to 70 Brix. In some embodiments, the system is configured to treat the viscous fluid to form a treated fluid having an acrylamide content of less than 2 μg/kg, a total furan content of less than μg/kg, a hydroxy methyl furan content of less than 5 ppm, and a 4-methylimidazole content of less than 0.0100 mg/kg, and a furfuryl alcohol content of less than 0.5 mg/kg.

Some embodiments are directed to a method of treating a viscous fluid including flowing the viscous fluid through a mixer such that the viscous fluid flows with a Reynolds number of at least 2200. In some embodiments, the method includes exposing the viscous fluid to UV light such that the viscous fluid receives a total dose of UV light of at least 500 mJ/cm². In some embodiments, the viscous fluid has a viscosity of 50 cP to 250 cP.

In some embodiments, the exposing the viscous fluid to UV light includes flowing the viscous fluid through a first UV chamber to expose the viscous fluid to a first dose of UV light of at least 250 mJ/cm².

In some embodiments, the method includes flowing the viscous fluid through a second mixer such that the viscous fluid flows with a Reynolds number of at least 2200.

In some embodiments, the viscous fluid flows from the mixer to the first UV chamber, and the viscous fluid flows from the first UV chamber to the second mixer.

In some embodiments, the exposing the viscous fluid to UV light includes flowing the viscous fluid through a second UV chamber to expose the viscous fluid to a second dose of UV light of at least 250 mJ/cm². In some embodiments, the total dose comprises the first dose and the second dose.

In some embodiments, the viscous fluid is a liquid sugar having a sugar content from 12 Brix to 70 Brix. In some embodiments, the viscous fluid is a liquid sugar having a sugar content from 60 Brix to 70 Brix. In some embodiments, the viscous fluid is a liquid sugar having a sugar content from 67 Brix to 68 Brix.

In some embodiments, the method is a continuous process configured to treat at least 1000 gallons of viscous fluid per hour.

Some embodiments are directed to a fluid treatment device including a first mixer configured to generate turbulent flow in a viscous fluid. In some embodiments, the device includes a first UV chamber configured to deliver a first dose of UV light to the viscous fluid. In some embodiments, the device includes a second mixer configured to generate turbulent flow in the viscous fluid. In some embodiments, the device includes a second UV chamber configured to deliver a second dose of UV light to the viscous fluid. In some embodiments, the first dose of UV light and the second dose of UV light together deliver at least 500 mJ/cm² of UV light.

In some embodiments, the first dose of UV light delivers at least 250 mJ/cm², and the second dose of UV light delivers at least 250 mJ/cm².

In some embodiments, the first mixer and the second mixer are each static mixers.

In some embodiments, the first UV chamber and the second UV chamber each comprise a UV lamp.

In some embodiments, the viscous fluid has a viscosity of at least 50 cP.

In some embodiments, the viscous fluid has a viscosity of at least 200 cP.

### BRIEF DESCRIPTION OF THE FIGURES

The accompanying drawings, which are incorporated herein and form part of the specification, illustrate the present disclosure and, together with the description, further serve to explain the principles of the disclosure and to enable a person skill in the relevant art to make and use the invention.

FIG. 1 illustrates a process flow diagram for systems according to some embodiments.

FIG. 2 illustrates a process flow diagram for systems according to some embodiments.

FIG. 3 illustrates a flow chart for methods according to some embodiments.

FIG. 4 illustrates a flow chart for methods according to some embodiments.

### DETAILED DESCRIPTION

Food and beverage products are often produced using various ingredients, including for example, viscous fluids (e.g., fluids having a viscosity of at least 150 cP). For example, many food and beverage products are produced using a liquid sugar. To ensure food and beverage safety, these viscous fluids must be treated before use in production to reduce or eliminate contaminants. Existing processes require heating the viscous fluid to high temperatures and maintaining the high temperature for extended periods. For example, thermal pasteurization requires heating the viscous fluid to over 230° F. and maintaining that temperature for at least 30 seconds. Not only is this energy intensive, it requires significant capital and operational costs. Additionally, after being heated, the viscous fluid may need to be cooled before use as an ingredient in a food or beverage. This may require either extended operation times to allow natural cooling or further equipment and energy costs to accelerate cooling (e.g., using refrigeration).

Although UV light can be used, for example, for treating low solids content and low viscosity fluids (e.g., fluids having a viscosity of less than about 50 cP) such as apple cider or apple juice, methods intended for treating such low-viscosity fluids fail to adequately treat fluids with higher solids content and higher viscosity. Because of the flow dynamics of fluids with higher viscosity and higher solids content, methods for UV-treating low-viscosity fluids are not effective treating high-viscosity fluids.

Embodiments described herein overcome these and other challenges by providing—among other benefits—systems and methods for non-thermal treatments of viscous fluids (e.g., liquid sugars) to remove contaminants. Moreover, embodiments described herein allow for non-thermal treatment of viscous fluids that do not adversely affect the quality attributes of the viscous fluid or the resulting food or beverage (e.g., taste, acidity, turbidity, color, etc.).

As shown throughout the figures, some embodiments are directed to systems and processes for non-thermal treatment of a viscous fluid. As used herein, the term “viscous fluid” means a fluid having a viscosity of at least 150 cP. For example, systems for treating the viscous fluid may include mixers and UV chambers. The mixers may generate perpendicular mixing to linear flow or turbulent flow in the viscous fluid before the viscous fluid passes through the UV chambers. Such turbulent flow may ensure that the viscous fluid is efficiently exposed to UV light to treat the viscous fluid.

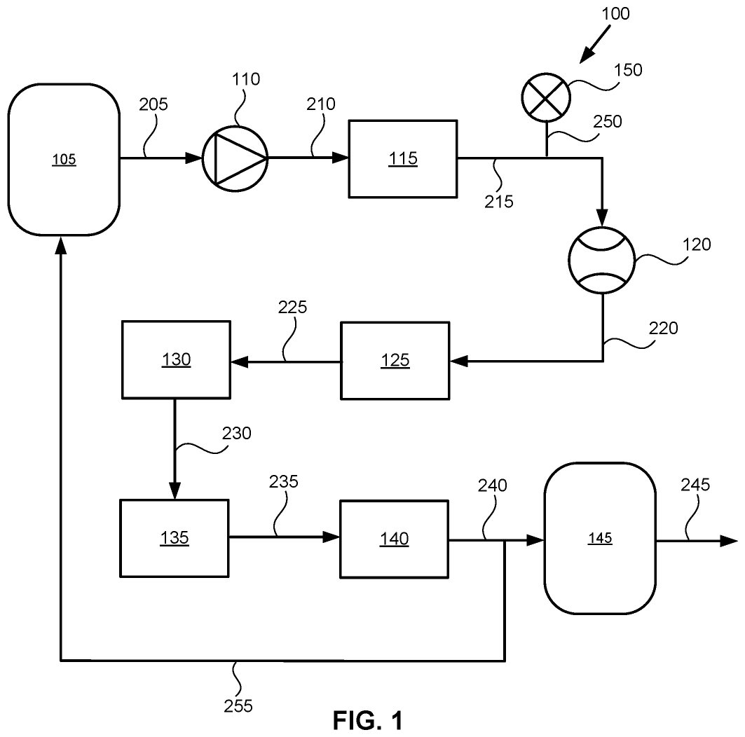

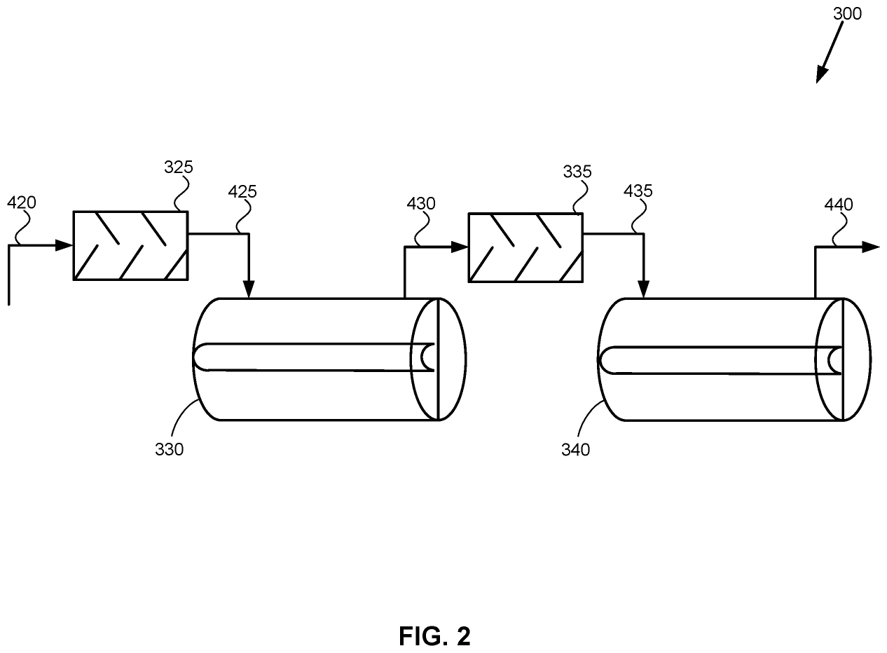

For example, FIGS. 1 and 2 show systems (e.g., system 100 and system 300) according to some embodiments. As shown in FIGS. 1 and 2, systems according to some embodiments may be configured to flow viscous fluid through the system and may include mixers (e.g., mixers 125, 135, 325, or 335) that increase the Reynolds number of the fluid flowing through the system to generate turbulent flow. The system may include UV chambers (e.g., UV chambers 130, 140, 330, or 340) that expose the viscous fluid flowing through the system to UV light to reduce contaminants in the viscous fluid. In some embodiments, the mixers mix the viscous fluid sufficient to achieve turbulent flow before the viscous fluid enters a UV chamber. In some embodiments, the mixers mix the viscous fluid sufficient to increase the Reynolds number of the viscous fluid to at least 2200 or improve perpendicular mixing versus the direction of flow. In some embodiments, system 100 or 300 may be used to treat viscous fluid using the UV chambers to remove contaminants and inactivate bacteria without requiring any thermal treatment. For example, in some embodiments, system 100 or 300 may be used to remove at least 95% (e.g., at least 99%) of contaminants and to inactivate at least 95% (e.g., at least 99%) of bacteria. In some embodiments, system 100 or 300 is used to remove 95% to 100% (e.g., 99% to 100%) of contaminants. In some embodiments, system 100 or 300 is used to inactivate 95% to 100% (e.g., 99% to 100%) of bacteria. In some embodiments, the microorganisms that are reduced and/or inactivated include one or more of bacteria of the genus Escherichia (e.g., Escherichia coli O157:H7 (ATCC 43894)); bacteria of the genus Bacillus (e.g., Bacillus atrophaeus (ATCC 9372) or Bacillus pumilus (ATCC 27142)); bacteria of the genus Alicyclobacillus (e.g., Alicyclobacillus spp. (ACB)); fungi of the genus Rhinocladia (e.g., Rhinocladia similis); yeast; mold; heat-resistant mold; and spoilage bacteria. As used herein the ATCC number refers to the number assigned to a specific organism strain by the American Type Culture Collection (“ATCC”) organization. Reducing and/or inactivating these microorganisms can reduce the chance of spoiled final products. For example, some of these microorganisms can survive heat pasteurization processes but cannot survive UV treatment according to embodiments disclosed herein. Additionally, in some embodiments, no contaminant by-products were produced in the viscous liquid during UV treatment according to methods disclosed here.

FIG. 1 shows a system 100 according to some embodiments. In some embodiments, system 100 includes untreated fluid tank 105, pump 110, filter 115, flow meter 120, mixer 125, UV chamber 130, mixer 135, UV chamber 140, and treated fluid tank 145, and temperature indicator 150. System 100 may include various inlets, pipes, and outlets. For example, in some embodiments, system 100 includes pipes 205, 210, 215, 220, 225, 230, 235, 240, and 250. In some embodiments, system 100 includes outlet 245. In some embodiments, system 100 includes recirculation pipe 255. In some embodiments, the system recirculates fluid until the UV chamber (e.g., UV chamber 130 or UV chamber 140) is ready to receive fluid (e.g., one or more lamps within each UV chamber is warmed up to deliver a desired UV intensity).

FIG. 2 shows a system 300 according to some embodiments, which may be an implementation of system 100. In some embodiments, system 300 includes static mixer 325, UV chamber 330, static mixer 335, and UV chamber 340. System 300 may include various inlets, pipes, and outlets. For example, in some embodiments, system 300 includes pipes 420, 425, 430, 435, and 440.

In some embodiments, untreated fluid tank 105 is used to store untreated viscous fluid. In some embodiments, the viscous fluid has a viscosity of at least 50 cP (e.g., at least 100 cP, at least 150 cP, at least 200 cP or at least 250 cP). In some embodiments, the viscous fluid has a viscosity of about 50 cP to about 300 cP (e.g., about 150 cP to about 300 cP, about 200 cP to about 250 cP or about 230 cP to about 250 cP). In some embodiments, the viscous fluid has an ultraviolet transmittance (“UVT”) of about 25% to about 50% (e.g., about 25% to about 35%). In some embodiments, the viscous fluid has a UVT of about 30%. In some embodiments, the viscous liquid has a viscosity of about 150 cP to about 250 cP and a UVT greater than 25%.

In some embodiments, the viscous fluid is liquid sugar formed from water and sugar. In some embodiments, the liquid sugar has a sugar content from about 12 Brix to about 70 Brix (e.g., about 30 Brix to about 70 Brix, about 60 Brix to about 70 Brix, about 65 Brix to about 68 Brix or about 67 Brix to about 68 Brix). In some embodiments, the liquid sugar has a sugar content of about 67.5 Brix. In some embodiments, the liquid sugar is suitable for use in beverages, including carbonated and non-carbonated beverages.

In some embodiments, untreated fluid tank 105 is a melting tank used to prepare the viscous fluid. For example, in embodiments where the viscous fluid is liquid sugar, untreated fluid tank 105 may be used to mix water and sugar to form the liquid sugar. In some embodiments, untreated viscous fluid may be transferred from untreated fluid tank 105 to mixer 125. In some embodiments, system 100 includes pump 110, filter 115, and flow meter 120 between untreated fluid tank 105 and mixer 125. In some embodiments, pump 110 pumps untreated viscous fluid from untreated fluid tank 105. In some embodiments, system 100 includes filter 115 for removing particulate matter. In some embodiments, filter 115 is configured to remove particles 5 micron and larger. System 100 may include flow meter 120 configured to control the flow rate of the viscous fluid flowing through the system. In some embodiments, systems described herein operated in a continuous manner. In some embodiments, flow meter 120 controls the flow rate of the viscous fluid such that the viscous fluid flows continuously. In some embodiments, the methods and systems described herein treat about 500 gallons to about 2500 gallons (e.g., about 1000 gallons to about 2000 gallons) of viscous fluid per hour.

System 100 may include at least one mixer (e.g., mixer 125 or mixer 135) that may increase the Reynolds number of the viscous fluid flowing through the system. For example, in some embodiments, the viscous fluid flowing into a mixer (e.g., mixer 125 or mixer 135) may flow in laminar flow. In some embodiments, the viscous fluid flowing into a mixer (e.g., mixer 125 or mixer 135) from pipes (e.g., pipe 220 or pipe 230) may be dominated by laminar flow. In some embodiments, the mixer (e.g., mixer 125 or mixer 135) may increase the Reynolds number of the viscous fluid such that the viscous fluid flowing out of the mixer may be dominated by turbulent flow. In some embodiments, the viscous fluid is dominated by turbulent flow when the Reynolds number is at least 2200. In some embodiments, the viscous fluid flowing out of the mixer has a Reynolds number of at least 2100 (e.g., at least 2200, at least 2500, at least 3000, or at least 4000). In some embodiments, the viscous fluid flowing out of the mixer has a Reynolds number of at least 2200.

In some embodiments, the system includes two mixers. In some embodiments, as shown in FIG. 1, system 100 includes mixer 125 and mixer 135. In some embodiments, as shown in FIG. 2, system 300 includes mixer 325 and mixer 335. Each mixer (e.g., mixers 125, 135, 325, or 335) may be any type of mixer suitable for generating turbulent flow in the viscous fluid. In some embodiments, as shown in FIG. 2, system 300 includes two static mixers (static mixer 325 and static mixer 335). Mixers 125 and 140 may also be static mixers, in some embodiments. In some embodiments, static mixers allow for reduction in required UV doses compared to other types of mixers.

In some embodiments, the system includes two UV chambers for treating viscous fluid flowing through the UV chambers. In some embodiments, the viscous fluid flowing through each UV chamber is characterized by at least partial turbulent flow (e.g., a Reynolds number greater than or equal to 2200). In some embodiments, the viscous fluid flowing through each UV chamber is dominated by turbulent flow. In some embodiments, as shown in FIG. 1, system 100 includes UV chamber 130 and UV chamber 140. In some embodiments, as shown in FIG. 2, system 300 includes UV chamber 330 and UV chamber 340. In some embodiments, the UV chambers are in series with one another to provide multiple, smaller doses of UV light, which can help prevent quality deterioration that can occur with higher, single doses. In some embodiments, each UV chamber includes at least one UV lamp (e.g., at least two UV lamps or at least three UV lamps). UV chamber 330 and UV chamber 340 may each be a barrel chamber with the at least one UV lamp positioned in the center. In some embodiments, each lamp is a medium pressure UV lamp. In some embodiments, one or more lamps operates at polychromatic wavelengths.

The dose delivered to the viscous fluid may be adjusted to account for specific conditions of the viscous fluid (e.g., turbidity or absorbance). Each UV chamber may deliver a dose of UV light to the viscous fluid flowing through the UV chamber. As used herein, a dose of UV light (mJ/cm²) is equal to the intensity of UV light (W/cm²) multiplied by the time of exposure (seconds). In some embodiments, each dose of UV light delivers at least about 150 mJ/cm² of UV light (e.g., at least about 200 mJ/cm², at least about 250 mJ/cm², at least about 300 mJ/cm², or at least about 400 mJ/cm², or at least about 500 mJ/cm²). In some embodiments, each dose of UV light delivers about 75 mJ/cm² to about 500 mJ/cm² (e.g., about 175 mJ/cm² to about 350 mJ/cm², or about 200 mJ/cm² to about 300 mJ/cm²). In some embodiments, each dose of UV light delivers about 250 mJ/cm². In some embodiments, the system delivers a total dose of UV light of about 150 mJ/cm² to about 1000 mJ/cm² of UV light (e.g., about 350 mJ/cm² to about 700 mJ/cm² or about 400 mJ/cm² or about 600 mJ/cm²). In some embodiments, the system delivers a total dose of UV light of about 500 mJ/cm².

In some embodiments, each UV chamber delivers an equal dose of UV light. In some embodiments, each UV chamber delivers a dose of about 250 mJ/cm². For example, in some embodiments, UV chambers 130 and 140 in system 100 (or UV chambers 330 and 340 in system 300) each deliver a dose of about 250 mJ/cm² such that system 100 delivers a total dose of about 500 mJ/cm².

In some embodiments, each UV chamber delivers unequal doses of UV light. In some embodiments, the total doses delivered by system is about 500 mJ/cm². For example, in some embodiments, UV chambers 130 and 140 in system 100 (or UV chambers 330 and 340 in system 300) each deliver different doses of UV light, but system 100 delivers a total dose of about 500 mJ/cm².

Each UV chamber may expose the viscous fluid to UV light for a predetermined time. In some embodiments, each UV chamber exposes the viscous fluid to UV light for at least 1 second. For example, in some embodiments, UV chambers 130 and 140 in system 100 (or UV chambers 330 and 340 in system 300) each expose the viscous fluid to UV light for at least 1 second (e.g., at least 2 second or at least 3 seconds). In some embodiments, UV chambers 130 and 140 in system 100 (or UV chambers 330 and 340 in system 300) each expose the viscous fluid to UV light for about 0.5 seconds to about 5 seconds (e.g., about 1 second to about 3 seconds, about 1 second to about 2 seconds or about 1 second to about 1.5 seconds). In some embodiments, UV chambers 130 and 140 in system 100 (or UV chambers 330 and 340 in system 300) each expose the viscous fluid to UV light for about 1.15 seconds.

In some embodiments, system 100 may include a recirculation line (e.g., recirculation pipe 255). In some embodiments, recirculation pipe 255 is used to recirculate viscous fluid exiting the UV chamber (e.g., UV chamber 140 or UV chamber 440) back to untreated fluid tank 105 for further treatment, as illustrated in FIG. 1. In some embodiments, recirculation pipe 225 reconnects between untreated fluid tank 105 and pump 110.

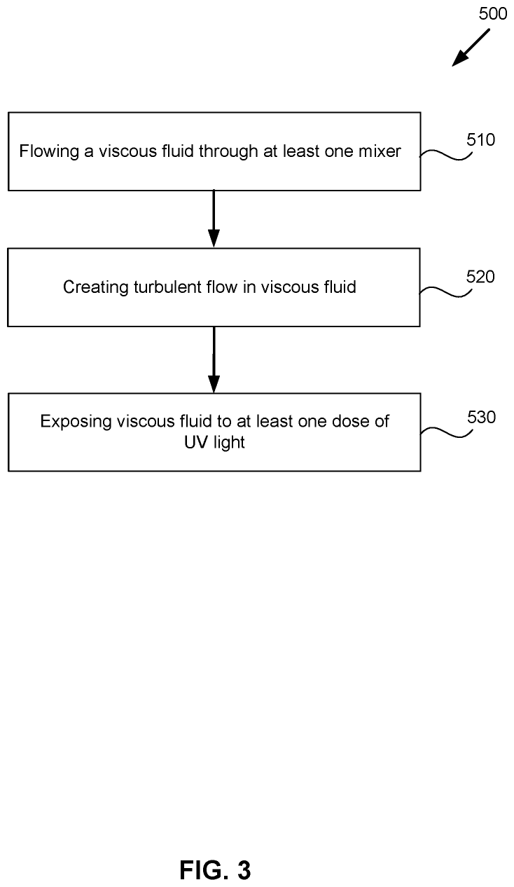

FIG. 3 shows a process 500 according to some embodiments. In some embodiments, process 500 may be performed on systems such as those shown in FIGS. 1 and 2. In some embodiments, at step 510, viscous fluid is flowed through at least one mixer (e.g., mixer 125, mixer 135, static mixer 325, or static mixer 335). In some embodiments, at step 520, the at least one mixer is used to create turbulent flow in the viscous fluid. In some embodiments, at step 530, after turbulent flow is generated in the viscous fluid, the viscous fluid is exposed to at least one dose of UV light. The at least one dose of UV light is described in detail above.

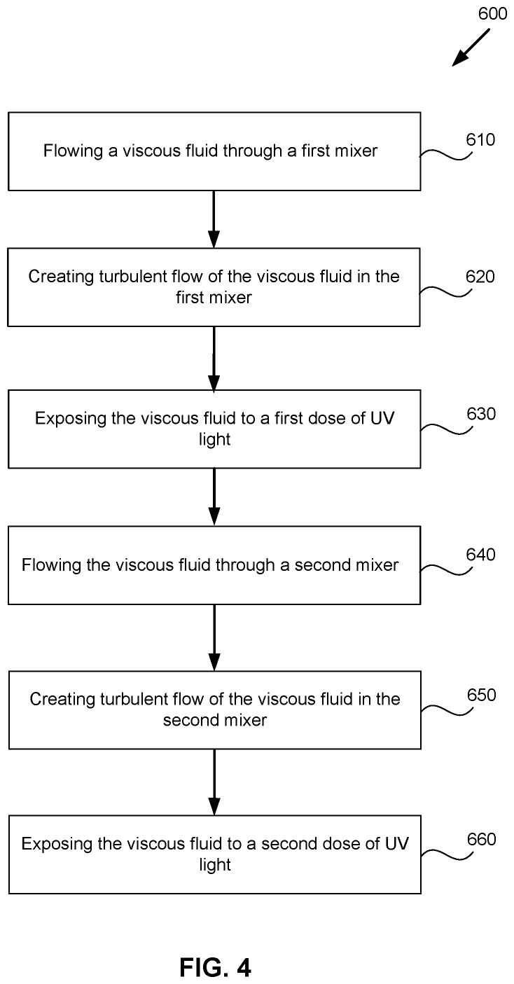

FIG. 4 shows a process 600 according to some embodiments. In some embodiments, process 600 may be performed on systems such as those shown in FIGS. 1 and 2. In some embodiments, at step 610, viscous fluid is flowed through a first mixer (e.g., mixer 125 or static mixer 325). In some embodiments, at step 620, first mixer creates turbulent flow in the viscous fluid. In some embodiments, at step 630, the viscous fluid is exposed to a first dose of UV light. In some embodiments, at step 640, the viscous fluid is flowed through a second mixer (e.g., mixer 135 or static mixer 335). In some embodiments, at step 650, the second mixer creates turbulent flow in the viscous fluid. In some embodiments, at step 660, the viscous fluid is exposed to a second dose of UV light.

As shown in the examples below, the methods and systems described herein may be used to treat viscous fluid such that contaminants are reduced. For example, as described above, the methods and systems described herein may be used to remove at least 95% (e.g., at least 99%) of contaminants and to inactivate at least 95% (e.g., at least 99%) of bacteria. For example, methods and systems described herein may be used to form a treated fluid having an acrylamide content of less than 2 μg/kg, a total furan content of less than μg/kg, a hydroxy methyl furan content of less than 5 ppm, and a 4-methylimidazole content of less than 0.0100 mg/kg, and a furfuryl alcohol content of less than 0.5 mg/kg.

### EXAMPLES

### Example 1

Various samples were tested for analytical and quality attributes. For example, pH, color, turbidity, and ash percent were tested for various liquid sugar samples having a sugar content of about 67.6 Brix to about 68 Brix. Table 1 summarizes the samples used for testing throughout the Examples.

TABLE 1

| | Treatment | Total UV dose | |
|---|---|---|---|
| Sample | type | (mJ/cm²) | Brix |
| A | untreated | 0 | 67.6 |
| B | thermal | 0 | 67.61 |
| C | UV | 173 | 67.93 |
| D | UV | 346 | 67.67 |
| E | UV | 500 | 67.7 |
| F | UV | 692 | 67.67 |

As shown in Table 1, Sample A was raw and untreated liquid sugar; Sample B was liquid sugar treated using a thermal processes, without UV treatment; and Samples C-F were liquid sugar treated using UV treatments according to some embodiments described herein. Samples C-F were treated using various total doses of UV light.

As shown in Table 2 below, analytical and quality attributes were tested, including pH, color, turbidity, and ash percent. Color was measured using the International Commission for Uniform Methods of Sugar Analysis (“ICUMSA”) scale. The ICUMSA scale defines pure, white sugar as a ICUMSA value of 45. Lower ICUMSA values correspond to less light absorption. Lightness values (L*) were measured for each sample. The lightness scale defines black at 0 and white at 100. Turbidity was measured according to the International Society of Beverage Technologists (“ISBT”). Ash is a measure of sugar quality, and the ash content includes organic and inorganic compounds.

TABLE 2

| | | Color | Lightness | Turbidity | |
|---|---|---|---|---|---|
| Sample | pH | ICUMSA | (L*) | ISBT | Ash % |
| A | 6.23 | 25 | 95.88 | 3 | 0.013 |
| B | 6.19 | 27 | 96.31 | 4 | 0.012 |
| C | 6.52 | 27 | 96.33 | 1 | 0.012 |
| D | 6.22 | 27 | 95.56 | 1 | 0.012 |
| E | 6.27 | 29 | 96.47 | <1 | 0.013 |
| F | 6.12 | 28 | 96.33 | <1 | 0.012 |

As shown in Table 2, the samples treated according to embodiments disclosed herein (i.e., Samples C-F) showed analytical and quality attributes similar to the thermally treated Sample B. For example, Samples C-F showed similar color, lightness, and ash values compared to Samples A and B. And Samples C-F showed reduced turbidity, which corresponds to reduced impurities.

As shown in Example 1, UV treatment according to some embodiments described herein can be used without negatively affecting the analytical and quality attributes of the viscous fluid, thereby effectively treating the viscous fluid without the higher cost, time, and energy consumption attendant to thermal treatment.

### Example 2

Microbial inoculation tests were performed on Samples C-F before and after UV treatment according to some embodiments disclosed herein. Before treatment, Samples C-F each included 4.6 log of B. pumilus (ATCC 27142). Samples C-F were treated using UV doses shown in Table 1 above. Table 3 shows the initial log count and final log count of B. pumilus (ATCC 27142) in the samples.

TABLE 3

| | Initial Log | Final Log |
|---|---|---|
| Sample | Count | Count |
| C | 4.6 | 0 |
| D | 4.6 | 0 |
| E | 4.6 | 0 |
| F | 4.6 | 0 |

Some bacteria, such as B. pumilus, show high resistance to UV light exposure. However, as shown in Table 3 above, at all tested UV doses, there was completed inactivation of B. pumilus (i.e., 4.6 log reduction was achieved).

As shown in Example 2, UV treatment according to some embodiments described herein can be used inactivate bacteria such as B. pumilus.

### Example 3

Contaminant tests were performed on liquid sugar samples having a sugar content of about 67.5 Brix (Samples G-J). As shown in Table 4, Sample G was treated using conventional thermal processes, without UV treatment, and Sampled H-J were treated using UV treatments according to some embodiments described herein.

TABLE 4

| | Treatment | UV dose |
|---|---|---|
| Sample | type | (mJ/cm²) |
| G | thermal | 0 |
| H | UV | 346 |
| I | UV | 692 |
| J | UV | 1000 |

As shown in Table 5 below, the samples were tested for various furan compounds, and the total furan concentration was tested. And as shown in Table 6 below, various other contaminants were tested.

TABLE 5

| Contaminant | Sample G | Sample H | Sample I | Sample J |
|---|---|---|---|---|
| Furan | <5 μg/kg | <5 μg/kg | <5 μg/kg | <5 μg/kg |
| 2-Methylfuran | <5 μg/kg | <5 μg/kg | <5 μg/kg | <5 μg/kg |
| 3-Methylfuran | <5 μg/kg | <5 μg/kg | <5 μg/kg | <5 μg/kg |
| 2-Ethylfuran | <5 μg/kg | <5 μg/kg | <5 μg/kg | <5 μg/kg |
| 2-Pentylfuran | <5 μg/kg | <5 μg/kg | <5 μg/kg | <5 μg/kg |
| 2,5- | <5 μg/kg | <5 μg/kg | <5 μg/kg | <5 μg/kg |
| Dimethylfuran | | | | |
| Total Furan | <30 μg/kg | <30 μg/kg | <30 μg/kg | <30 μg/kg |

As shown in Table 5, all tested furan compounds were below the detection limit of the equipment used for testing. Additionally, the total furan concentration was below the detection limit of the equipment used for testing.

TABLE 6

| Contaminant | Sample G | | Sample H | | Sample I | | Sample J | |
|---|---|---|---|---|---|---|---|---|
| Acrylamide | <2 | μg/kg | <2 | μg/kg | <2 | μg/kg | <2 | μg/kg |
| Hydroxy Methyl | <5 | ppm | <5 | ppm | <5 | ppm | <5 | ppm |
| Furfural (HMF) | | | | | | | | |
| 4-Methylimidazole | <0.0100 | mg/kg | <0.0100 | mg/kg | <0.0100 | mg/kg | <0.0100 | mg/kg |
| Furfuryl alcohol | <0.5 | mg/kg | <0.5 | mg/kg | <0.5 | mg/kg | <0.5 | mg/kg |

As shown in Table 6, all other tested compounds were below the detection limit of the equipment used for testing.

As shown in Example 3, UV treatment according to some embodiments described herein can be used to significantly reduce contaminants such as those described above.

### Example 4

Liquid sugar was used to produce four beverages (Beverages 1, 1′, 2, and 2′). Beverages 1 and 1′ were made using the same process and ingredients, except Beverage 1 used thermally treated liquid sugar, and Beverage 1′ used UV treated liquid sugar. Beverages 2 and 2′ were made using the same process and ingredients, except Beverage 2 used thermally treated liquid sugar, and Beverage 2′ used UV treated liquid sugar. Beverages 1′ and 2′ were each treated with a total dose of 500 mJ/cm² of UV light. Each beverage was tested for various sensory attributes (e.g., appearance liking, overall flavor liking, sweetness liking, and mouthfeel liking). Beverages were tested by consumers, and the consumers rated the beverage based on the various sensory attributes. Scoring was done based on a Hedonic rating scale from 1 to 9, where 1 means the consumer disliked extremely and 9 means the consumer liked extremely.

TABLE 7

| | | | Overall | | |
|---|---|---|---|---|---|
| | | Appearance | Flavor | Sweetness | Mouthfeel |
| Beverage | Process | Liking | Liking | Liking | Liking |
| 1 | Thermal | 8 | 7.5 | 7.3 | 7.6 |
| 1′ | UV | 7.9 | 7.5 | 7.2 | 7.5 |
| 2 | Thermal | 8.2 | 7.8 | 7.5 | 7.5 |
| 2′ | UV | 8.2 | 7.7 | 7.5 | 7.9 |

As shown in Table 7, the beverages (1′, 2′) with UV-treated liquid sugar scored very similarly to beverages with thermally treated liquid sugar (1, 2). Accordingly, liquid sugar treated according to embodiments disclosed herein can be used in products without affecting the consumer experience of the product.

As shown in Example 4, UV treatment according to some embodiments described herein can be used without affecting sensory attributes (e.g., appearance liking, overall flavor liking, sweetness liking, and mouthfeel liking).

As used herein, the term “laminar flow” means fluid flow in which the fluid travels smoothly or in regular paths. Laminar flow may be defined in terms of the Reynolds number. In some embodiments, fluid flow described herein may be considered to flow with laminar flow when the Reynolds number of the fluid flowing through a pipe is less than 2100.

As used herein, the term “turbulent flow” means fluid flow in which fluid travels in an unstable path. Turbulent flow may be defined in terms of the Reynolds number. In some embodiments, fluid flow described herein may be considered to flow with turbulent flow when turbulent flow begins to develop. In some embodiments, fluid flow described herein may be considered to flow with turbulent flow when the Reynolds number of the fluid flowing through a pipe is greater than 2100.

As used herein, when the term “about” is used in describing a value or an end-point of a range, the disclosure should be understood to include the specific value or end-point referred to. As used herein, the term “about” may include±10%.

It is to be appreciated that the Detailed Description section, and not any other section, is intended to be used to interpret the claims. Other sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The above examples are illustrative, but not limiting, of the present disclosure. Other suitable modifications and adaptations of the variety of conditions and parameters normally encountered in the field, and which would be apparent to those skilled in the art, are within the spirit and scope of the disclosure.

References in the specification to “some embodiments” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The breadth and scope of the present disclosure should not be limited by any of the above-described exemplary embodiments, but should be defined only in accordance with the claims and their equivalents.

What is claimed is:

1. A system for treating a viscous fluid, the system comprising:
a mixer configured to receive the viscous fluid and to generate turbulent flow of the viscous fluid;
a UV chamber configured to receive the viscous fluid from the mixer and to expose the viscous fluid to a dose of UV light, the dose being at least 250 mJ/cm²,
wherein the viscous fluid has a viscosity of at least 50 cP.

2. The system of claim 1, wherein the viscous fluid has a viscosity of 50 cP to 250 cP.

3. The system of claim 1, further comprising a second mixer configured to receive the viscous fluid from the UV chamber and to generate turbulent flow of the viscous fluid; and
a second UV chamber configured to receive the viscous fluid from the second mixer and to expose the viscous fluid to a second dose of UV light to produce a treated viscous fluid, the second dose being at least 250 mJ/cm².

4. The system of claim 3, wherein the UV chamber is configured to expose the viscous fluid to UV light for about 1 second to about 5 seconds, and wherein the second UV chamber is configured to expose the viscous fluid UV light for about 1 second to about 5 seconds.

5. The system of claim 3, wherein mixer and the second mixer are each a static mixer.

6. The system of claim 1, wherein system is configured to treat the viscous fluid to form a treated fluid having an acrylamide content of less than 2 μg/kg, a total furan content of less than μg/kg, a hydroxy methyl furan content of less than 5 ppm, and a 4-methylimidazole content of less than 0.0100 mg/kg, and a furfuryl alcohol content of less than 0.5 mg/kg.

7. The system of claim 1, wherein the viscous fluid is a liquid sugar, and
wherein the system further comprises a melting tank, the melting tank configured to form the liquid sugar from water and sugar.

8. The system of claim 1, wherein the viscous fluid is a liquid sugar having a sugar content from 60 Brix to 70 Brix.

9. The system of claim 8, wherein the viscous fluid has an ultraviolet transmittance of about 25% to about 50%.

10. The system of claim 1, wherein the viscous fluid is liquid sugar having a sugar content from 67 Brix to 68 Brix.

11. The system of claim 1, wherein the system is configured to maintain the viscous fluid at a Reynolds number of at least 2200 through the UV chamber.

12. The system of claim 1, further comprising:
a melting tank configured to form the viscous fluid, wherein the viscous fluid is a liquid sugar formed from water and sugar, and wherein the liquid sugar has a sugar content from 60 Brix to 70 Brix,
wherein system is configured to treat the viscous fluid to form a treated fluid having an acrylamide content of less than 2 μg/kg, a total furan content of less than μg/kg, a hydroxy methyl furan content of less than 5 ppm, and a 4-methylimidazole content of less than 0.0100 mg/kg, and a furfuryl alcohol content of less than 0.5 mg/kg.

13. A method of treating a viscous fluid, the method comprising:
flowing the viscous fluid through a mixer such that the viscous fluid flows with a Reynolds number of at least 2200;
exposing the viscous fluid to UV light such that the viscous fluid receives a total dose of UV light of at least 500 mJ/cm²,
wherein the viscous fluid has a viscosity of 50 cP to 250 cP.

14. The method of claim 13, wherein the exposing the viscous fluid to UV light comprises:
flowing the viscous fluid through a first UV chamber to expose the viscous fluid to a first dose of UV light, the first dose being at least 250 mJ/cm².

15. The method of claim 14, further comprising:
flowing the viscous fluid through a second mixer such that the viscous fluid flows with a Reynolds number of at least 2200.

16. The method of claim 15, wherein the viscous fluid flows from the mixer to the first UV chamber, and wherein the viscous fluid flows from the first UV chamber to the second mixer.

17. The method of claim 14, wherein the exposing the viscous fluid to UV light comprises:
flowing the viscous fluid through a second UV chamber to expose the viscous fluid to a second dose of UV light, the second dose being at least 250 mJ/cm²,
wherein the total dose comprises the first dose and the second dose.

18. The method of claim 13, wherein the viscous fluid is a liquid sugar having a sugar content from 60 Brix to 70 Brix.

19. The method of claim 13, wherein the viscous fluid is a liquid sugar having a sugar content from 67 Brix to 68 Brix.

20. The method of claim 13, wherein the method is a continuous process configured to treat at least 1000 gallons of viscous fluid per hour.

21. A fluid treatment device, the device comprising:
a first mixer configured to generate turbulent flow in a viscous fluid;
a first UV chamber configured to deliver a first dose of UV light to the viscous fluid;
a second mixer configured to generate turbulent flow in the viscous fluid;
a second UV chamber configured to deliver a second dose of UV light to the viscous fluid,
wherein the first dose of UV light and the second dose of UV light together deliver at least 500 mJ/cm² of UV light.

22. The fluid treatment device of claim 21, wherein the first dose of UV light delivers at least 250 mJ/cm², and wherein the second dose of UV light delivers at least 250 mJ/cm².

23. The fluid treatment device of claim 21, wherein the first mixer and the second mixer are each static mixers.

24. The fluid treatment device of claim 23, wherein the first UV chamber and the second UV chamber each comprise a UV lamp.

25. The fluid treatment device of claim 21, wherein the viscous fluid has a viscosity of at least 50 cP.
